(19) United States
(12) Patent Application Publication
(10) Pub. No.: US 2013/0029933 A1
(43) Pub. Date: Jan. 31, 2013

(54) Hydrogel for Natural Cosmetic Purposes

(71) Applicants: Iris Schnitzler, Bonn (DE); Christian Hausen, St. Katharinen (DE); Christina Klein, Mayen (DE)

(72) Inventors: Iris Schnitzler, Bonn (DE); Christian Hausen, St. Katharinen (DE); Christina Klein, Mayen (DE)

(73) Assignee: LTS LOHMANN THERAPIE-SYSTEME AG, Andernach (DE)

(21) Appl. No.: 13/639,065

(22) PCT Filed: Apr. 11, 2011
(86) PCT No.: PCT/EP2011/001788

§ 371 Date: Oct. 2, 2012

(30) Foreign Application Priority Data
Apr. 13, 2010 (DE) DE 102010014869.5

(51) Int. Cl. A61K 8/73; A61Q 19/00

(52) U.S. Cl. 514/54

(57) ABSTRACT

The subject matter of the present invention is a hydrogel which includes a combination of at least four polysaccharides. These polysaccharides are those which are in the natural state and which are not produced, altered or modified by chemical reactions. The combination of konjac mannan, xanthan gum, pullulan and carrageenan and optionally sclerotium gum produces, within certain concentration ranges, a stable, slightly sticky, neutral smelling, flexible and transparent hydrogel which can be used as a cosmetic product for skin care.

The present invention relates to a hydrogel, in particular in the form of a plaster. The invention further relates to the preparation of this hydrogel and to its use for cosmetic purposes.

The cosmetics market is increasingly asking for “natural products”. This is to be understood as meaning that the constituents used in cosmetic formulations consist of natural raw materials, i.e. raw materials not prepared by chemical synthesis and/or by chemical modification of natural substances. Obtaining such (natural) substances should also have little impact on nature, e.g. as a result of sustainable ecological cultivation.

Moreover, the user should not come into contact unnecessarily with synthetic, harmful or allergy-causing constituents.

Nevertheless, the user makes high demands on the appearance and the odour, the effect and also stability of the product.

Known hydrogels are based on polyacrylic acids produced by polymerization on an industrial scale, or salts thereof, or comprise other gel formers produced by chemical synthesis, such as polyvinylpyrrolidone (PVP), polyvinyl alcohol (PVA), HPMC (hydroxypropylmethyl-cellulose—a partially synthetic substance) carbomer etc.

Furthermore, on account of their susceptibility to the build-up of germs, such hydrogels comprise considerable amounts of preservatives, usually a mixture of benzyl alcohol, phenoxyethanol, parabens, chlorine-containing substances or others.

It was therefore an object of the invention to develop a hydrogel which makes do without chemically produced or altered gel formers, uses mild preservatives which are accepted by the customary labels such as “Ökotest”, “Ecocert”, NaTrue, BDIH standard etc., and have a visually and olfactory pleasing appearance and retain these properties over a prolonged period, preferably at least 12 months.

In this description, “chemically produced gel formers” or “chemically altered gel formers” are to be understood as meaning those synthetic or natural substances which are produced, altered or modified by chemical reactions (such as polymerization, polyadditions, esterification, etc.).

The object is achieved by a hydrogel which comprises—besides the obligatory water—a combination of at least four polysaccharides. Furthermore, the hydrogel can comprise at least one organic acid.

The four polysaccharides are those which are in a “natural state” and are formed by known, “biological” (also microbiological) production processes. These polysaccharides are thus not synthetically produced, altered or modified by chemical reactions (such as polymerization, polyadditions, esterification etc.). Only mechanical and thermal processes are used for the purpose of comminuting, separating, transporting and purifying the polysaccharides from their natural sources. These include filtration, sedimentation, centrifugation, drying. Thermal processes can also be used for these purposes (e.g. for distillation, rectification, mass separation by means of membranes). In each case, there is no formation of covalent bonds.

The combination according to the invention of these at least four polysaccharides comprises:
a) konjac mannan,
b) xanthan gum,
c) pullulan, and
d) carrageenan.

Within certain concentration ranges of these polysaccharides, this leads to synergy effects, which form a stable, slightly adhesive, flexible and transparent hydrogel with a neutral odour. This hydrogel has adequate cut resistance, meaning that a cosmetic product—for example for skincare, can be produced by appropriate further processing.

Konjac mannan is a natural polysaccharide from the tuber of the konjac plant (Amorphophallus Konjac), which thrives in subtropical regions, primarily in Indonesia, in south western China, sometimes in Japan. Production takes place by the pulverization of the tubers, removal of foreign bodies, washing of the flour, sedimentation with alcohol and subsequent extraction. It has the CAS Reg. No. [37220-17-0].

Xanthan gum is a microbial, anionic polysaccharide which is secreted from Xanthomonas campestris under suitable cultivation conditions. It has the CAS Reg. No. [11138-66-2] and is commercially available, specifically from a product series with the trade name Keltrol C G. Particular preference is given to Keltrol CG-SFT.

Pullulan is an extracellular polysaccharide of the yeast-like fungus Aureobasidium pullulans (synonyms: Pullularia pullulans, Dematium pullulans), which is widespread in stretches of standing water. Pullulan is a homopolysaccharide with D-glucose as the only building block. It has the CAS Reg. No. [9057-02-7].

Carrageenan is the extract from dried red algae of the species Chondrus crispus and Gigartina stellata. The gel-forming κ fraction (kappa fraction) consists of d-galactose-4-sulphate and 3,6-anhydro-α-d-galactose, which is alternatingly glycosidically bonded in the 1,3 and 1,4 position. Carrageenan has the CAS Reg. No. [9000-07-1] and the preferred κ-carrageenan has the CAS Reg. No. [11114-20-8]. The i fraction (iota fraction) of carrageenan (CAS Reg. No. [9062-07-1]) may also be used, in combination with the κ fraction if desired. The λ fraction (lambda fraction) of carrageenan is not suitable.

In a further embodiment the hydrogel may also comprise a fifth polysaccharide, namely “sclerotium gum” which is also called amigel. This is a polysaccharide which is prepared from the fungus Sclerotium rolfsii by fermentation.

In the hydrogel according to the invention, the concentrations of konjac mannan, xanthan gum and pullulan are between 0.1 and 2%, preferably between 0.15 and 0.5%. If sclerotium gum is used as a fifth polysaccharide, this is likewise present in a concentration between 0.1 and 2%, preferably between 0.15 and 0.5%. The concentration of carrageenan is between 1 and 20%, preferably between 5 and 15% and particularly preferably between 8 and 12%. This % by weight data is based on the hydrogel, i.e. the water-containing end product, which can optionally also comprise further ingredients, in particular cosmetic active ingredients.

As preservative for stabilizing against germs, fungi and yeasts, at least one organic acid is used. This may be formic acid, benzoic acid, dehydracetic acid, acetic acid, fumaric acid, 4-hydroxybenzoic acid, hydroxysuccinic acid, lactic acid, propionic acid, salicylic acid, sorbic acid etc., and also salts and mixtures thereof. Preference is given to benzoic acid, dehydracetic acid, lactic acid and sorbic acid, in particular a mixture thereof. 2-Phenoxyethanol is also suitable as preservative.

The hydrogel can also comprise at least one technical auxiliary, which improves e.g. its mechanical properties. These include softeners, moisture regulators, antioxidants, pH buffers, dyes, binders, surfactants, viscosity improvers.

To develop a pleasant odour and to increase the cosmetic effect on the skin (increase in skin moisture, increase in elasticity, wrinkle smoothing, supplying the skin with essential substances (minerals, vitamins, fatty acids, lipids etc.)), the hydrogel can comprise at least one fragrance and/or at least one cosmetic active ingredient.

The fragrances include odourants, i.e. uniform, defined chemical compounds with an odour. These include natural aroma substances, nature-identical aroma substances, synthetic aroma substances, aroma extracts, reaction aromas and smoke aromas. Mixtures such as essential oils are also included here.

The cosmetic active ingredients include skin oils, skincare agents and skin protectants which are known to the person skilled in the art, for example from DE 102 41 597.

The process for the preparation of the hydrogel consists, in a first step, in heating water to at least 40° C. This water (“phase 1”) may also already contain further constituents of the hydrogel, for example at least one technical auxiliary and/or at least one cosmetic active ingredient and/or a preservative.

In a second step, a combination of 0.1-2% by weight of konjac mannan, 0.1-2% by weight of xanthan gum and 0.1-2% by weight of pullulan, and also 0.1-2% by weight of sclerotium gum if used, is added, with the formation of an aqueous solution, to the heated water from the first step. In order to achieve complete dissolution of these polysaccharides (“phase 2”), it may be necessary to stir and heat further, but not above 95° C.

During the second step, at least one technical auxiliary, at least one cosmetic active ingredient, at least one fragrance and/or at least one preservative can also be added. At the end of the second step, 1-20% by weight of carrageenan (“phase 4”) are added.

The solution obtained at the end of the second step and still at least 40° C. hot is then, in a third step, applied to a substrate—preferably in a constant layer thickness—and converted to a solid hydrogel with cooling. Siliconized PET carrier films, for example, can be used as substrate. In the finished product, such a film then serves as a covering layer (release liner).

After cooling to room temperature, a stable hydrogel is obtained which can be further processed using standard commercial processes such as cutting, die-cutting, laminating, packing etc.

In one preferred variant of the preparation process, the hydrogel is also covered with a nonwoven fabric during cooling and before reaching room temperature.

In a second variant of the preparation process “phase 2” (=the mixture of polysaccharides) and “phase 4” the composition containing carrageenan) are combined before they are added to the water-containing “phase 1”. The addition takes place by sucking in the dry, pulverulent mixture of phases 2 and 4. Subsequently the components of phase 3 (=preservative, technical auxiliaries, e.g. dexpanthenol, org. acids) and phase 5 (cosmetic active ingredients, e.g. plant extracts; fragrances, e.g. ethereal oils) may likewise be added to the mixture of phase 1 and phases 2/4 concomitantly.

The hydrogel according to the invention will be illustrated in more detail by the preparation examples below.

### EXAMPLE 1

### Preparation of a Hydrogel for Natural Cosmetics

| Component | % by wt. |
|---|---|
| Phase 1: | |
| Water | 57.2 |
| Glycerol 86% | 8.0 |
| Phase 2: | |
| Konjac mannan | 0.2 |
| Xanthan gum | 0.2 |
| Pullulan | 0.2 |
| Phase 3: | |
| Dexpanthenol 75% | 6.7 |
| Organic acid | 0.5 |
| Phase 4: | |
| Carrageenan | 10.0 |
| Sucrose | 10.0 |
| Phase 5: | |
| Plant extracts and essential oils | 7.0 |

Phase 1 is heated to 60° C. and, in succession, phase 2, phase 3, phase 4 and finally phase 5 are slowly added with stirring until complete homogeneity. The resulting solution is coated onto a siliconized PET liner in a coating box, heated at 50° C., with a gap width of 500 μm and then covered with a 100% viscous nonwoven. The fractions of the components given in the tabular overview of the constituents of the hydrogel are given, as also throughout the entire description—in % by weight, based on the hydrogel.

After cooling to room temperature, a stable hydrogel is obtained, which can be cut into pieces or die-cut as desired. Packaging then takes place in a water-impermeable composite packaging material made of PE/paper with an aluminium barrier layer.

### EXAMPLE 2

### Preparation of a Hydrogel for Natural Cosmetics

| Component | % by wt. |
|---|---|
| Phase 1: | |
| Water | 60.6 |
| Glycerol 86% | 8.0 |
| Phases 2 and 4: | |
| Konjac mannan | 0.3 |
| Xanthan gum | 0.3 |
| Sclerotium gum | 0.3 |
| Pullulan | 0.3 |
| i-carrageenan | 6.0 |
| Sucrose | 10.0 |
| Phases 3 and 5: | |
| Dexpanthenol 75% | 6.7 |
| Organic acid | 0.5 |
| Plant extracts and essential oils | 7.0 |

Phase 1 is heated to 60° C. and then initially the mixture of phase 2 und phase 4 is added, then the mixture of phase 3 and phase 5. Further processing is as per example 1.

### EXAMPLE 3

### Preparation of a Hydrogel for Natural Cosmetics

| Component | % by wt. |
|---|---|
| Phase 1: | |
| Water | 79.3 |
| Glycerol 86% | 4.0 |
| Phases 2 and 4: | |
| Konjac mannan | 1.0 |
| Xanthan gum | 0.1 |
| Pullulan | 0.1 |
| K-carrageenan | 2.0 |
| Sucrose | 1.0 |
| Phases 3 and 5: | |
| Dexpanthenol 75% | 2.0 |
| organic acid | 0.5 |
| Plant extracts and essential oils | 10.0 |

### Processing as per Example 2.

The hydrogel according to the invention can be used for the cosmetic treatment of the human skin, in particular the facial skin. Since the hydrogel is slightly adhesive, if it is situated on a carrier film as a result of the production, it is removed from this and placed onto the area of human skin in question, if necessary with the application of slight pressure.

The hydrogel can be used—depending on the particular at least one cosmetic active ingredient present therein—for cooling, for calming the skin, for smoothing wrinkles, for stabilizing the water balance in the skin. The use of Sclerotium gum resulted in a significant improvement of the stability of the hydrogel, especially in the cut edge region, which is due to an increase in cohesion and thus also in cut firmness.

1. Hydrogel comprising a combination of
a. 0.1-2% by weight of konjac mannan,
b. 0.1-2% by weight of xanthan gum,
c. 0.1-2% by weight of pullulan
d. 1-20% by weight of carrageenan and
e. water.

2. Hydrogel according to claim 1, wherein said hydrogel additionally comprises 0.1-2% by weight of sclerotium gum.

3. Hydrogel according to claim 1, wherein said hydrogel further comprises 0.1-5% by weight of a preservative.

4. Hydrogel according to claim 1, wherein said hydrogel comprises at least one cosmetic active ingredient from the group of skin oils, skincare agents and skin protectants.

5. Hydrogel according to claim 1, wherein said hydrogel comprises at least one technical auxiliary and/or a fragrance.

6. Hydrogel according to claim 3, wherein the preservative comprises at least one organic acid.

7. Process for the preparation of a hydrogel comprising, in a first step, heating water to at least 40° C., in a second step adding a combination of 0.1-2% by weight of konjac mannan, 0.1-2% by weight of xanthan gum, 0.1-2% by weight of pullulan and 1-20% by weight of carrageenan to form an aqueous solution, and in a third step applying the solution obtained in the second step to a substrate and converting the applied solution to a solid hydrogel with cooling.

8. Process according to claim 7, wherein 0.1-5% by weight of at least one preservative is added to the water and/or to the aqueous solution.

9. Process according to claim 7, wherein said process further comprises placing a nonwoven fabric layer onto the hydrogel during cooling.

10. Process according to claim 7, wherein the combination of konjac mannan, xanthan gum, pullulan and carrageenan additionally comprises from 0.1 to 2% by weight of Sclerotium gum.

11. A cosmetic treatment for human skin formed from a hydrogel comprising a combination of 0.1-2% by weight of konjac mannan, 0.1-2% by weight of xanthan gum, 0.1-2% by weight of pullulan and 1-2 0% by weight of carrageenan and water.

12. Hydrogel stabilizer comprising sclerotium gum, wherein the resulting stabilized hydrogel exhibits increased cohesion.

13. Hydrogel according to claim 6, wherein the organic acid is benzoic acid, dehydracetic acid, lactic acid, sorbic acid or a mixture thereof.

14. A cosmetic treatment for human skin as claimed in claim 11, wherein in said skin is facial skin.
